(19) United States
(12) Patent Application Publication
(10) Pub. No.: US 2004/0073182 A1
(43) Pub. Date: Apr. 15, 2004

(54) Absorbent core, a process of making the absorbent core and a disposable absorbent article with the absorbent core

(76) Inventors: Carlos Da Silva Macedo Jr, Sao Jose Dos Campos (BR); Eduardo Cezar Andreo Aledo, Sao Jose Do Campos (BR); Altair De Paula Vitor, Sao Jose Do Campos (BR); Francisco J Vladivia Hernandez, Campos (ES)

Correspondence Address: PHILIP S. JOHNSON, JOHNSON & JOHNSON, ONE JOHNSON & JOHNSON PLAZA, NEW BRUNSWICK, NJ, 08933-7003, US

(21) Appl. No.: 10/399,248

(22) PCT Filed: Oct. 17, 2001
(86) PCT No.: PCT/BR01/00130

(30) Foreign Application Priority Data
Oct. 19, 2000 (BR) PI 00004935-2

(51) Int. Cl. A61F 13/15; A61F 13/20

(52) U.S. Cl. 604/385.01

(57) ABSTRACT

The present disclosure relates to an absorbent core and to the process of making the absorbent core. The process involves no loss of material during manufacturing of absorbent core. The absorbent core has an anatomic format enabling a comfortable use with high absorption power, which may be used in various disposable absorbent products for urinary incontinence, disposable diapers or sanitary napkin.

### FIELD OF THE INVENTION

The present invention relates to an absorbent core and to a process of making the absorbent core for use in disposable absorbent products for urinary incontinence, disposable diapers and sanitary napkins.

### BACKGROUND OF THE INVENTION

The absorbent core of known disposable absorbent products is usually formed of a cellulose fiber pulp, often comprising super-absorbent polymers capable of forming a gel in contact with liquids as well. Other absorbent cores are also known such as tissue paper, peat moss, artificial fibers and foams and like.

Processes for preparing and cutting absorbent cores from a continuous sheet of absorbent material are also known in the art. The cuts are made by knives, ultrasound, laser or water under pressure and the like, in order to obtain absorbent products cut from the continuous sheet of absorbent material either along a longitudinal or transverse direction of the continuous sheet.

The disadvantages of previous art cutting processes, due to the curved side border shape of these absorbent cores plus the disposition of the absorbent cores in the longitudinal direction, reside in a high compulsory loss of material, compromising noticeably the productivity.

### SUMMARY OF THE INVENTION

It is an object of this invention to provide an absorbent core and a process of making this absorbent core where there is no compulsory loss of material during the manufacturing of the absorbent core.

In accordance with present invention there has been provided a process of making an absorbent core that is useful in disposable absorbent articles, the process comprising steps of:

providing a continuous sheet of absorbent material having a longitudinal length, a first side edge and an opposite side edge defining therebetween a transverse width;

feeding the continuous sheet of absorbent material into a cutting means in the direction of the longitudinal length;

cutting the continuous sheet of absorbent material in an alternating pattern including a first cutting line and a second cutting line, each cutting line extending from the first side edge to the opposite second side edge to substantially transversely cut the continuous sheet of absorbent material, thereby defining a plurality of consecutive absorbent pieces, each consecutive absorbent piece having a length equal to the transverse width of the continuous sheet of absorbent material, wherein a portion of the first cutting line adjacent the first side edge is longitudinally offset from at least a portion of the first cutting line adjacent the opposite second side edge and a portion of the second cutting line adjacent the second side edge is longitudinally offset from at least a portion of the second cutting line adjacent the opposite first side edge, and wherein each respective offset is in the direction of the longitudinal length;

juxtaposing at least two adjacent consecutive absorbent pieces obtained through the cutting of the continuous sheet of absorbent material.

In accordance with the present invention there has also been provided an absorbent core comprising a first absorbent piece and a second absorbent piece, each absorbent piece being formed from a sheet of absorbent material and each absorbent piece having a respective first distal end region and a respective second opposite distal end region, the first distal end region having a width that is greater than a width of the second distal end region, the first absorbent piece and the second absorbent piece being substantially of identical length and having substantially identical shape when the first absorbent piece is in an inverted position at 180° relative to the second absorbent piece, the first absorbent piece being juxtaposed over the second absorbent piece such that at least a portion of the first distal end region of the first absorbent piece is vertically aligned with at least a portion of the second distal end region of the second absorbent piece.

### BRIEF DESCRIPTION OF THE DRAWINGS

This invention will be next described below in more details, based on the following figures, which shows:

FIG. 1—is a top view of the absorbent core of this invention;

FIG. 2—is a side view of the absorbent core shown in FIG. 1;

FIG. 3—is a side view of the absorbent core shown in FIGS. 1 and 2, in a position equivalent to that used by the user, slightly bent; and

FIG. 4—is a top view of the cut configuration of the absorbent core shown in FIGS. 1 to 3.

### DETAILED DESCRIPTION OF THE DRAWINGS

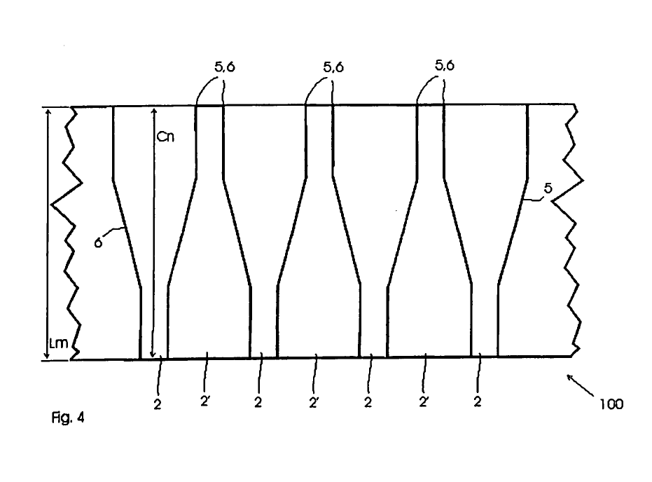

The present invention is directed to an absorbent core, and to a method of forming the absorbent core. More particularly, the present invention is directed to a process of making an absorbent core that has no compulsory loss of material during the process. According to a first embodiment and as shown in FIG. 4, the process to obtain the absorbent core of this invention comprises the following steps; feeding a continuous sheet of absorbent material 100, the sheet of absorbent material having a longitudinal extension length, a first side edge and an opposite second side edge defining therebetween a width Lm. The step of feeding the continuous sheet of absorbent material 100 is accomplished in the direction of its length.

The sheet of absorbent material 100 is continuously and alternately cut along a first cutting line and a second cutting line. The first cutting line extends from the first side edge of the absorbent material across the width of the absorbent material to the opposite, second side edge. Similarly, the second cutting line extends from the first side edge of the absorbent material across the width of the absorbent material to the opposite, second side edge. The first cutting line is longitudinally spaced apart from the second cutting line.

The step of cutting the continuous sheet of absorbent material 100 defines at least two consecutive absorbent pieces including a first absorbent piece 2 and a second absorbent piece 2′. Each cutting line forms one of two opposite lateral edges, respectively, of the two consecutive and coinciding absorbent pieces. The first cutting line defining the first lateral edge 5 and the second cutting line defining the second lateral edge 6 of the consecutive absorbent pieces, the first and the second edges lateral edges 5,6 coinciding on adjacent consecutive pieces 2,2′.

As shown in FIG. 2. each absorbent piece 2,2′ has a longitudinal dimension Cn that extends in the same direction and has the same value as the width Lm of the continuous sheet of absorbent material 100 as shown in FIG. 4. Thus a longitudinal axis of the absorbent piece is transverse to the direction of feeding the continuous sheet of absorbent material 100 and accordingly, the length of the absorbent pieces corresponds to the transverse width of the continuous sheet of absorbent material 100.

A portion of the first cutting line adjacent the first side edge of the sheet of the absorbent material 100 is longitudinally offset from at least a portion of the first cutting line adjacent the opposite second side edge of the absorbent material 100. Similarly, a portion of the second cutting line adjacent the second side edge of the absorbent material 100 is longitudinally offset from at least a portion of the second cutting line adjacent the opposite first side edge of the absorbent material 100. Each respective offset is in the direction of the longitudinal length of the absorbent material 100. Each cutting line preferably has the identical shape except that the first cutting line is a mirror image of the second cutting line, i.e. if one cutting line is rotated 180° about a center point on its longitudinal axis it would be identical to and capable of being superimposed onto the adjacent cutting line.

In a preferred embodiment, each cutting line has a substantially straight portion extending across a portion of the width of the absorbent material 100 adjacent each respective first side edge and opposite second side edge of the continuous sheet of absorbent material 100 and a central region that extends obliquely across the balance of the width of the continuous sheet of absorbent material 100.

Referring to FIG. 1, each absorbent piece 2, 2′ has a first lateral edge 5 and an opposite second lateral edge 6. The first and second lateral edges 5, 6 define therebetween a width of the absorbent piece. Each absorbent piece, 2,2′ has a first distal end region adjacent the first side edge and a second distal end region adjacent the second lateral edge and a central region intermediate the first distal end region and the second distal end region. Since a portion of the first cutting line adjacent the first side edge is longitudinally offset from at least a portion of the first cutting line adjacent the opposite second side edge and a portion of the second cutting line adjacent the second side edge is longitudinally offset from at least a portion of the second cutting line adjacent the opposite first side edge, wherein each respective offset is in the direction of the longitudinal length, each resulting absorbent piece 2,2′ has a varying width along its length. More particularly, for a given absorbent piece 2, the first distal end region has a relatively narrow width relative to its opposite second distal end region and for an adjacent absorbent piece 2′, the first distal end region has a relatively wider width relative to its opposite second distal end region. Accordingly, the widths of the first distal end regions of adjacent absorbent pieces 2, 2′ will alternate from narrow to wide to narrow et. seq. In addition, the widths of the second distal end regions of adjacent absorbent pieces 2, 2′ will also alternate from narrow to wide to narrow et. seq., in a manner opposite to that of the first distal end regions.

As shown in FIGS. 1 and 2, the next step in the process comprises juxtaposing at least two consecutive absorbent pieces 2,2′ one on top of another to form the absorbent core of the invention. A first absorbent piece 2 is juxtaposed over an adjacent piece 2′ such that at least a portion of the first distal end of the first absorbent piece 2 is vertically aligned with at least a portion of the first distal end of the second absorbent piece 2′ as shown in FIG. 1.

Thus, in the subject process of this invention, the sheet cutting stage of absorbent material 100 generates a plurality of absorbent pieces 2,2′ laid in an adjacent way, side by side, with their edges of opposite extremities coinciding with the first and second lateral edges 5,6 of the continuous sheet of the absorbent material 100, presenting therefore the same orientation and opposing directions of the sheet 100 positioning. The continuous sheet of absorbent material 100 may be cut by any cutting means generally known in the art such as by knives, ultrasound, laser, water under pressure and the like and combinations thereof.

This process eliminates material wastage, i.e. compulsory loss, as there are no interstitial spaces between the two absorbent pieces 2,2′. We notice that the existence of interstitial spaces between the absorbent pieces 2,2′, cause material wastage during the productive process, generating a surplus which is of no use and must be eliminated.

In a preferred embodiment, each one of the absorbent pieces 2,2′ has one first extremity portion with length and width presenting substantially the same figure, and a second portion of the extremity which presents a width equivalent to approximately one third of the length presented by the first portion and the length equivalent to that presented by the first portion. Both the first and the second extremity portions are put together, at their length, through a central portion possessing a substantially trapezoidal format, presenting lateral edges in the central region that are substantially inclined in relation to the length of the absorbent pieces 2,2′ and which unite the lateral edges of the first and second parts of the extremity.

Each of the absorbent pieces 2,2′ preferably have a caliper (or thickness) of less that 5 mm and in a preferred embodiment, the combined caliper of the absorbent core in the central region 10 is less than 5 mm. Thus, the absorbent cores of the present invention present a considerably reduced thickness compared to the smaller width, which enables the manufacturing of urinary incontinence products, diapers and sanitary napkins considerably thinner, making them more comfortable, discreet and attractive.

So as to form the absorbent core 1, the first absorbent piece 2′ is put together over a second absorbent piece 2, in the opposite direction, so that the first part of the extremity of an absorbent piece and the second portion of the extremity of the other absorbent piece remain juxtaposed. The association region of the absorbent pieces is represented in the figures by number 4. The connection of the absorbent pieces 2,2′ may be done by gluing them or through an adhesive element, such as tapes, according to the needs or convenience for production and operation. Alternatively, the absorbent pieces may contain fusible fibers and the connection may be done by conventional thermobonding techniques.

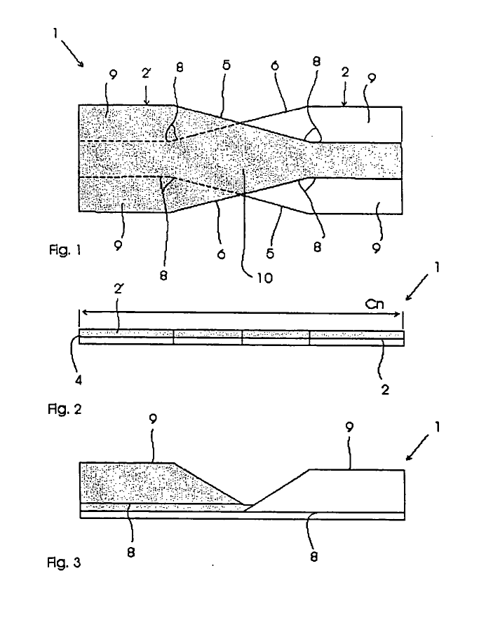

Referring to FIGS. 1 and 3, the union of absorbent pieces 2,2′, in an opposite direction, as mentioned above, leads to the existence within the whole absorbent core core 1, of four external regions 9 symmetrically located within the external quadrants of the absorbent core 1, each of the external regions 9 possessing only one segment of the absorbent pieces 2,2′. The delimitation region between the external regions 9 above mentioned and a central region 10, where the two absorbent pieces 2,2′ are juxtaposed, form a bending region, as it allows the external regions 9, of smaller thickness, to bend in comparison to the central region 10, such bend occurs exactly in the bending region, which possesses the format of two bending lines 8. This way, the central region 10, of the absorbent core 1 is considerably rigid and possesses a great absorbent capacity, while the external regions 9 have a smaller absorbent power, but present more flexibility.

Therefore, the central part of the absorbent core 1 possesses converging inclined extremity lines, while in the extremity portions, the extremity lines are substantially parallel. It is of course possible to use a plurality of juxtaposed pieces, without causing the invention to suffer substantial alterations that make it considerably different from the preferential example described herein.

The juxtaposition may be obtained through the absorbent pieces 2,2′ of the sheet of absorbent material 100 themselves or through the association with other juxtaposed sheets and simultaneous to the sheet of absorbent material 100. The format of the absorbent pieces 2,2′ may vary, such as bottle, pin, etc.

When the user wears the urinary incontinence product, or diaper or sanitary napkin containing the absorbent core 1 in question, the external region of each one of the absorbent pieces 2,2′ tends to bend, as from the bending lines 8, causing these bending lines 8 to form “walls” going against user's groin and internal part of the thighs, blocking the region and preventing user's bodily fluid to leak, ensuring efficiency and well being when using the absorbent element 1. Apart from ensuring the efficiency of the absorbent element 1, the external regions 9, as they have reduced thickness and are flexible, provide comfort when using, without practically being noticed, which would not occur should these external regions 9 be manufactured using a rigid, hard or very thick material.

As advantages, the invention presents a process where there is no compulsory loss of material during the manufacturing of absorbent core, apart from the absorbent core present an anatomic format, with high absorption capacity due to the fact that it presents a greater quantity of absorbent material in the user's bodily fluid discharge region, and may be interchangeably used in products for urinary incontinence, disposable diapers or in sanitary napkins. Furthermore, it enables a comfortable use, considering that the layers of absorbent material have reduced thickness.

After describing an example of preferred embodiments, it must be understood that the scope of this invention encompasses other possible variations, limited only by the tenor of the attached claims, including therein the possible equivalents.

1. A process of making an absorbent core that is useful in disposable absorbent articles, the process comprising steps of:
providing a continuous sheet of absorbent material having a longitudinal length, a first side edge and an opposite second side edge defining therebetween a transverse width;
feeding the continuous sheet of absorbent material into a cutting means in the direction of the longitudinal length;
cutting the continuous sheet of absorbent material in an alternating pattern including a first cutting line and a second cutting line, each cutting line extending from the first side edge to the opposite second side edge to substantially transversely cut the continuous sheet of absorbent material, thereby defining a plurality of consecutive absorbent pieces, each consecutive absorbent piece having a length equal to the transverse width of the continuous sheet of absorbent material, wherein a portion of the first cutting line adjacent the first side edge is longitudinally offset from at least a portion of the first cutting line adjacent the opposite second side edge and a portion of the second cutting line adjacent the second side edge is longitudinally offset from at least a portion of the second cutting line adjacent the opposite first side edge, and wherein each respective offset is in the direction of the longitudinal length;
juxtaposing at least two adjacent consecutive absorbent pieces obtained through the cutting of the continuous sheet of absorbent material.

2. An absorbent core obtained through the process defined in claim 1.

3. An absorbent core comprising a first absorbent piece and a second absorbent piece, each absorbent piece being formed from a sheet of absorbent material and each absorbent piece having a respective first distal end region and a respective second opposite distal end region, the first distal end region having a width that is greater than a width of the second distal end region, the first absorbent piece and the second absorbent piece being substantially of identical length and having substantially identical shape when the first absorbent piece is in an inverted position at 180° relative to the second absorbent piece, the first absorbent piece being juxtaposed over the second absorbent piece such that at least a portion of the first distal end region of the first absorbent piece is vertically aligned with at least a portion of the second distal end region of the second absorbent piece.

4. The absorbent core according to claim 3, wherein the absorbent core has a length extending from the first distal end region to the second opposite distal end region and a central region that is formed by the first absorbent piece and the second absorbent piece, the central region extending along at least one half of the length of the absorbent core.

5. The absorbent core according to claim 4, wherein the absorbent core has a flexible region extending laterally outward from at least a portion of the central region, the flexible region being more flexible than the central region.

6. The absorbent core according to claim 5, wherein the flexible region is formed by either the first absorbent piece or the second absorbent piece, but not both the first and second absorbent pieces.

7. A disposable absorbent article comprising a body fluid permeable layer adapted to contact a user's body in use, a body fluid impermeable layer adapted to face a user's garment in use and an absorbent core intermediate the body fluid permeable layer and body fluid impermeable layer, wherein the absorbent core as defined in the claim 4.
